(19) United States
(12) Patent Application Publication
(10) Pub. No.: US 2016/0143864 A1
(43) Pub. Date: May 26, 2016

(54) EXTENDED-RELEASE PHARMACEUTICAL COMPOSITIONS OF METOPROLOL

(71) Applicant: SUN PHARMACEUTICAL INDUSTRIES LIMITED, Mumbai, Maharashtra (IN)

(72) Inventors: Sandeep Kumar VATS, Sonipat City, Haryana (IN); Balaram MONDAL, East Midnapore, West Bengal (IN); Kalaiselvan RAMARAJU, Tiruchirappalli, Tamil Nadu (IN); Romi Barat SINGH, Varanasi, Uttar Pradesh (IN); Ajay Kumar SINGLA, Gurgaon, Haryana (IN)

(21) Appl. No.: 14/904,167

(22) PCT Filed: Jul. 9, 2014
(86) PCT No.: PCT/IB2014/062981

§ 371 Date: Jan. 11, 2016

(30) Foreign Application Priority Data
Jul. 9, 2013 (IN) 2051/DEL/2013
May 1, 2014 (IN) 1181/DEL/2014

(51) Int. Cl. A61K 31/138; A61K 9/48

(52) U.S. Cl. CPC A61K 31/138; A61K 9/4808

(57) ABSTRACT

The present invention relates to timed extended-release pharmaceutical compositions comprising metoprolol and an extended-release polymer. The pharmaceutical compositions of the present invention exhibit an in-vivo lag time of at least 2 hours as and a Tₘₐₓ of more than 8 hours. Further, said pharmaceutical compositions provide an in-vitro release of metoprolol over a period of at least 20 hours.

### FIELD OF THE INVENTION

The present invention relates to timed extended-release pharmaceutical compositions comprising metoprolol and an extended-release polymer. The pharmaceutical compositions of the present invention exhibit an in-vivo lag time of at least 2 hours and a Tₘₐₓ of more than 8 hours. Further, said pharmaceutical compositions provide an in-vitro release of metoprolol over a period of at least 20 hours.

### BACKGROUND OF THE INVENTION

Coordinating biological rhythms (chronobiology) with medical treatment is called chronotherapy (Narayan et al., International Journal of Medicine and Pharmaceutical Research, 1(2):226-234, 2013). Human circadian rhythm is based on the sleep-activity cycle. The release of a number of hormones depends on this cycle, e.g., estrogen and progesterone are released by the brain in the morning, while melatonin and cortisol are released during sleep. These variations tend to influence the therapeutic efficacy of the drug. Blood pressure and heart rate are highest from 6:00 a.m. to 12:00 p.m. Hence, there is a need for a composition which can achieve the desired plasma levels of a cardiovascular drug at the time of day when blood pressure and heart rate are highest.

Beta-blockers are an important class of cardiovascular drugs. Metoprolol is a beta-blocker that is typically prescribed for the treatment of hypertension, angina pectoris, and stable symptomatic heart failure. It preferentially acts on beta₁ adrenoreceptors, which predominate in the cardiac muscles.

U.S. Publication No. 2012/0070472 discloses a chronotherapeutic composition comprising metoprolol tartarate and Eudragit® wherein metoprolol is completely released in 12 hours (in-vitro). It discloses a pharmaceutical composition with an in-vivo peak-to-trough ratio of greater than 4.

The present invention provides timed extended-release pharmaceutical compositions of metoprolol, wherein the pharmaceutical compositions of the present invention exhibit a lag time of at least 2 hours, and a Tₘₐₓ of more than 8 hours. Further, said pharmaceutical compositions provide an in-vitro release of metoprolol over a period of at least 20 hours.

### SUMMARY OF THE INVENTION

The pharmaceutical composition of the present invention is administered once daily at bedtime (approximately 9 p.m. to 10 p.m.) to a subject. The pharmaceutical composition provides a high plasma concentration of metoprolol during the early morning hours (approximately 4 a.m. to 12 p.m.) due to a Tₘₐₓ shift, thereby maintaining therapeutic plasma concentration throughout the day until the next dose is administered. Hence, the present invention provides synchronization of the drug release with the circadian rhythm of the body. The timed extended-release pharmaceutical compositions of the present invention exhibit a lag time of at least 2 hours and a Tₘₐₓ of more than 8 hours. Further, said pharmaceutical compositions provide an in-vitro release of metoprolol over a period of at least 20 hours.

### BRIEF DESCRIPTION OF THE FIGURES

FIG. 1 shows the plasma concentration of metoprolol following oral dosing at about 9:00 p.m. of timed extended-release capsule 100 mg vs. Toprol-XL® 100 mg.

### DETAILED DESCRIPTION OF THE INVENTION

According to a first aspect of the present invention, there is provided a timed extended-release pharmaceutical composition comprising:
a) a core comprising metoprolol;
b) an extended-release coating comprising an extended-release polymer; and
c) an outer coating comprising an enteric polymer
   wherein the pharmaceutical composition exhibits the following in-vitro dissolution profile, when measured in United States Pharmacopeia (USP) type 2 dissolution apparatus, with a paddle rotation at 50 rpm, at a temperature of 37° C.±0.5° C. in 500 mL of 0.1N HCl for 4 hours, followed by 900 mL of pH 7.5 phosphate buffer: (a) not more than about 15% of the total amount of metoprolol is released in 4 hours; and (b) more than 70% of the total amount of metoprolol is released in 20 hours to 24 hours.

The term “timed extended-release”, as used herein, means that the composition is timed to release metoprolol after a lag time of at least 2 hours, followed by an extended-release of metoprolol. The timed extended-release coatings are in particular prepared by applying an inner coating of an extended-release polymer onto a core, and an outer coating of an enteric polymer or a blend of an enteric polymer and an extended-release polymer. Alternatively, the extended-release cores may be further coated with metoprolol which may then be subsequently coated with an enteric polymer. The timed extended-release includes pulse-release pharmaceutical compositions. Pharmaceutical compositions of the present invention provide in-vitro release of metoprolol over a period of at least 20 hours, in particular 20 hours to 24 hours.

The term “metoprolol”, as used herein, includes metoprolol and its pharmaceutically acceptable salts. The pharmaceutically acceptable salts include succinate, fumarate, tartrate, citrate, pamoate, and mandelate. Metoprolol and its pharmaceutically acceptable salts may be either in their racemic form or as a pure enantiomer.

The term “core”, as used herein, may be a matrix core or a coated core. It may be in the form of pellets, granules, spheres, or mini-tablets. Coated cores may be prepared by coating metoprolol, optionally along with other pharmaceutically acceptable excipients, onto an inert bead. Optionally, a seal coat layer may be present between the inert bead and said coating layer comprising metoprolol. The inert bead may be water-soluble, water-swellable, or water-insoluble. Examples of water-swellable cores include microcrystalline cellulose spheres such as Celphere®. Examples of water-soluble cores include sugar spheres made of glucose, mannitol, lactose, xylitol, dextrose, or sucrose. Examples of water-insoluble cores include glass beads or silicon dioxide beads. Alternatively, the core may be a matrix core, formulated by mixing metoprolol, optionally with other pharmaceutically acceptable excipients, followed by granulation, direct compression, or extrusion-spheronization.

According to another embodiment of this aspect, the pharmaceutical composition provides: (a) not more than about 15% of the total amount of metoprolol is released in 4 hours; (b) less than about 60% of the total amount of metoprolol is released in 12 hours; and (c) more than 70% of the total amount of metoprolol is released in 20 hours to 24 hours.

According to a second aspect of the present invention, there is provided a timed extended-release pharmaceutical composition comprising:
a) a core comprising metoprolol;
b) an extended-release coating comprising an extended-release polymer; and
c) an outer coating comprising an enteric polymer
   wherein the pharmaceutical composition provides a Tₘₐₓ of more than 8 hours when administered to healthy human subjects.

The term “Tₘₐₓ” refers to the time at which the peak plasma level concentration of metoprolol is attained in a healthy human subject following administration of the metoprolol pharmaceutical composition.

According to one embodiment of this aspect, the pharmaceutical composition further provides an in-vivo lag time of at least 2 hours.

“Lag time” refers to the time between administration of a metoprolol pharmaceutical composition to healthy human subjects and the first quantifiable plasma level concentration of metoprolol in the plasma concentration versus time curve under fed conditions. The percentage drug release of not more than 15% in 0.1N HCl under in-vitro conditions is an indication of lag time under in-vivo conditions. The current commercially available metoprolol extended-release tablet Toprol-XL® composition does not exhibit such a lag time. This lag time is crucial for chronotherapeutic release profile. In particular, the in-vivo lag time provided by the timed extended-release capsule is at least 2 hours to 4 hours.

According to another embodiment of this aspect, the pharmaceutical composition further provides an in-vivo peak to trough ratio of drug plasma level of less than 4 under steady-state.

Peak-to-trough ratio of less than 4 indicates lesser fluctuation in a metoprolol plasma concentration at steady state. The term “peak-to-trough ratio” refers to the ratio of the maximum plasma concentration to the minimum plasma concentration in a dosing interval at steady-state.

According to yet another embodiment of this aspect, the pharmaceutical composition further provides Cₘₐₓ at a value between about 15.00 ng/mL to about 158.00 ng/mL in the plasma after administration of the metoprolol timed extended-release capsule to healthy human subject.

The term “Cₘₐₓ” refers to the maximum concentration of metoprolol in the plasma following administration of the metoprolol pharmaceutical composition to healthy human subjects.

According to another embodiment of this aspect, the pharmaceutical composition further provides AUC at a value between about 559.00 ng·hr/mL to about 9192.00 ng·hr/mL after administration of the metoprolol timed extended-release capsule to healthy human subjects.

The term “AUC” refers to the area under the time/plasma concentration curve after administration of the metoprolol pharmaceutical composition to healthy human subjects.

According to another embodiment of the above aspects, the timed extended-release pharmaceutical composition further comprises a timed immediate-release pharmaceutical composition.

According to another embodiment of the above aspects, the timed immediate-release pharmaceutical composition comprises:
a) a core comprising metoprolol; and
b) an outer coating comprising an enteric polymer.

According to another embodiment of the above aspects the timed extended-release pharmaceutical composition further comprises:
a) a first outer coating layer over the extended-release coating layer comprising metoprolol; and
b) a second outer coating layer over the first outer coating layer comprising an enteric polymer.

According to another embodiment of the above aspects, the core is a matrix core comprising metoprolol and optionally one or more pharmaceutically acceptable excipients.

According to another embodiment of the above aspects, the core is a coated core comprising:
a) an inert bead;
b) optionally, a seal coat layer over the inert bead; and
c) a coating layer comprising metoprolol over the inert bead of step a) or the coated bead of step b).

According to another embodiment of the above aspects, the extended-release coating is about 5% to about 15% based on the weight of the drug coated core.

According to yet another embodiment of the above aspects, the extended-release polymer is a water-soluble polymer, a water-insoluble polymer, or a mixture thereof.

According to another embodiment of the above aspects, the extended-release coating comprising a water-insoluble polymer further comprises a pore-former.

According to another embodiment of the above aspects, the extended-release coating comprises the water-insoluble polymer and the pore-former in a ratio of about 50:50 to about 99:1.

According to another embodiment of the above aspects, the extended-release coating comprises the water-insoluble polymer and the pore-former in a ratio of about 75:25 to about 95:5.

According to another embodiment of the above aspects, the enteric polymer is selected from the group comprising hydroxypropylmethyl cellulose acetate succinate, hydroxypropylmethyl cellulose phthalate, cellulose acetate phthalate, methacrylic acid copolymer, or mixtures thereof.

According to another embodiment of the above aspects, the enteric polymer is one or more methacrylic acid copolymer selected from the group comprising Eudragit® L 30 D-55, Eudragit® L 100-55, Eudragit® L 100, Eudragit® S 100, Eudragit® L 12,5, Eudragit® S 12,5, or Eudragit® FS 30 D.

According to another embodiment of the above aspects, the methacrylic polymer is Eudragit® L 30 D-55.

According to another embodiment of the above aspects, the methacrylic polymer is Eudragit® FS 30 D.

According to another embodiment of the above aspects, the methacrylic polymer is Eudragit® S 100.

According to another embodiment of the above aspects, the methacrylic polymer is Eudragit® L 100.

According to another embodiment of the above aspects, the methacrylic polymer is a combination of Eudragit® S 100 and Eudragit® L 100.

According to another embodiment of the above aspects, Eudragit® S 100 and Eudragit® L 100 are present in a ratio of about 1:1 to about 5:1.

The term “timed immediate-release”, as used herein, means that the composition is timed to release metoprolol after a lag time of at least 2 hours when administered to a subject, followed by an immediate-release of metoprolol. The timed immediate-release pharmaceutical composition comprises coated cores produced by applying an enteric polymer coating onto the immediate-release core comprising metoprolol.

Extended-release polymers used herein include water-soluble polymers, water-insoluble polymers, or mixtures thereof.

Water-soluble polymers include hydroxypropylmethyl cellulose having an apparent viscosity ranging from 80 to 120,000 cP (2% in water at 20° C.), e.g., K100, K4M, K15M, K100M, E4M, and E10M; hydroxypropyl cellulose, e.g., HPC-H, HPC-M, HPC-HF, HPC-HXF; polyethylene glycol (molecular weight of about 3000 or above); poly(ethylene oxide), e.g., PEO-27, PEO-18, PEO-15, PEO-8, PEO-4, Polyox® WSR-1105, and Polyox® WSR-303; hydroxyethyl cellulose; carboxymethyl cellulose; xanthan gum; starch; polyvinyl pyrrolidone; or mixtures thereof.

Water-insoluble polymers include cellulose ethers, e.g., ethylcellulose; cellulose esters, e.g., cellulose acetate; polymethacrylic acid esters copolymers, e.g., Eudragit® NE 30 D and Eudragit® NE 40 D; aminoalkyl methacrylate copolymers, e.g., Eudragit® RL 100, Eudragit® RL PO, Eudragit® RS PO, and Eudragit® RS 100; copolymers of polyvinyl acetate and polyvinyl pyrrolidone, e.g., Kollidon® SR; or mixtures thereof. In particular, the extended-release polymer is a water-insoluble polymer. More particularly the extended-release polymer is ethyl cellulose. The extended-release coating comprising a water-insoluble polymer further comprises a pore-former selected from the group comprising low viscosity grade hydroxypropylmethyl cellulose having an apparent viscosity of less than 100 cP (2% in water at 20° C.), e.g., K3, E5, E15, and E50; sodium alginate; sugars and sugar alcohols, e.g., sucrose, dextrose, lactose, maltitol, and lactitol; low molecular weight polyethylene glycol (molecular weight of less than 3000); polyvinyl alcohol; polyvinyl pyrrolidone; or mixtures thereof. The water-insoluble polymer and the pore former are present in a ratio of about 50:50 to about 99:1, in particular, from about 75:25 to about 95:5.

Enteric polymers, used herein, include hydroxypropylmethyl cellulose acetate succinate; hydroxypropylmethyl cellulose phthalate, e.g., hypromellose phthalate-55; cellulose acetate phthalate; methacrylic acid copolymer, e.g., Eudragit® L 30 D-55, Eudragit L 100-55, Eudragit L 100, Eudragit S 100, Eudragit L 12,5, Eudragit S 12,5 or Eudragit® FS 30 D; or mixtures thereof. The amount of an enteric polymer may vary from 1% to 70% by weight of the total composition. In particular, the enteric polymer is Eudragit® L 30 D-55, Eudragit® FS 30 D, Eudragit® L 100, Eudragit® S 100, or a combination of Eudragit® S 100 and Eudragit® L 100 in a ratio of about 1:1 to about 5:1.

Pharmaceutical compositions used herein may be in the form of capsules or tablets. Coated cores may be filled directly into a capsule shell, or compressed into a tablet. Further, capsules may be filled with a single type of coated cores comprising timed extended-release cores. Alternatively, capsules may be filled with two types of coated cores comprising timed extended-release cores and timed immediate-release cores. The ratio of timed extended-release cores to timed immediate-release cores may vary from about 1:100 to about 100:1. In particular, the ratio is from about 50:50 to about 100:1.

The pharmaceutical composition may further comprise other pharmaceutically acceptable excipients. Examples of pharmaceutically acceptable excipients include binders, diluents, lubricants/glidants, surfactants, or mixtures thereof.

Examples of binders include methyl cellulose, hydroxypropyl cellulose (HPC-L), carboxymethyl cellulose sodium, hydroxypropylmethyl cellulose, polyvinylpyrrolidone, or mixtures thereof.

Examples of diluents include lactose, calcium carbonate, calcium phosphate, calcium sulfate, microcrystalline cellulose, cellulose powdered, fructose, lactitol, mannitol, sorbitol, starch, sucrose, or mixtures thereof.

Examples of lubricants or glidants include colloidal silicon dioxide, stearic acid, magnesium stearate, calcium stearate, talc, hydrogenated castor oil, sucrose esters of fatty acid, microcrystalline wax, yellow beeswax, white beeswax, or mixtures thereof. They may be added intragranularly as well as extragranularly.

Examples of surfactants include sodium lauryl sulfate, sodium dodecyl sulfate, ammonium lauryl sulfate, benzalkonium chloride, alkyl poly(ethylene oxide), copolymers of poly(ethylene oxide) and poly(propylene oxide) commercially known as poloxamers or poloxamines, polyvinyl alcohol (PVA), fatty alcohols, polyoxyethylene alkyl ether, polyoxyethylene alkylaryl ether, polyethylene glycol fatty acid ester, alkylene glycol fatty acid mono ester, sucrose fatty acid ester, and sorbitan fatty acid mono ester, sorbitol monolaurate (Span® 20 or Span® 80), polyoxyethylene sorbitan fatty acid ester (polysorbates), or mixtures thereof.

The coatings of the present invention comprise excipients selected from the group comprising plasticizers, binders, opacifiers, anti-tacking agents, anti-foaming agents, film-forming polymers, colors, or mixtures thereof. Organic or aqueous solvents may be used during the coating process. Solvents may be selected from the group comprising water, acetone, isopropyl alcohol, ethanol, isopropyl acetate, methylene chloride, or mixtures thereof. In particular, isopropyl and water may be used for extended release coating in a ratio of 60:40 to 100:1.

Examples of plasticizers include propylene glycol, triethyl citrate, tributyl citrate, dibutyl sebacate, acetyl tributyl citrate, glyceryl monostearate, triacetin, polyethylene glycol, diethyl phthalate, acetylated monoglycerides, diacetylated monoglyceride, cetyl alcohol, or mixtures thereof.

Examples of opacifiers include titanium dioxide, silicon dioxide, talc, calcium carbonate, behenic acid, or mixtures thereof.

Examples of anti-tacking agents include talc, colloidal silicon dioxide, or mixtures thereof.

Examples of anti-foaming agents include silicon based surfactants, e.g., simethicone; vegetable oils; waxes; hydrophobic silica; polyethylene glycol; or mixtures thereof.

Coloring agents may be selected from FDA approved colorants such as iron oxide, lake of tartrazine, allura red, titanium dioxide, or mixtures thereof.

Examples of film-forming polymers include hydroxypropylmethyl cellulose, methyl cellulose, carboxymethyl cellulose, hydroxyethyl cellulose, polyethylene glycol, polyvinyl alcohol, or mixtures thereof. Alternatively, commercially available coating compositions comprising film-forming polymers marketed under various trade names, such as Opadry®, may also be used for coating.

Coating may be carried out by using any conventional coating techniques known in the art, such as spray coating in a conventional coating pan or fluidized bed processor, or dip coating.

The term “about”, as used herein, refers to any value which lies within the range defined by a variation of up to ±10% of the value.

The following examples illustrate the invention but are not to be construed as limiting the scope of the invention.

### EXAMPLES

### Example 1

### Timed Extended-Release Cores

| | quantity/capsule |
|---|---|
| Ingredients | (mg) |
| Metoprolol succinate | 190.00 |
| Opadry ® | 19.00 |
| Sugar spheres | 150.00 |
| Isopropyl alcohol | q.s. |
| Purified water | q.s. |
| Extended-Release Coating | |
| Ethocel ® 20 cps | 18.10 |
| Opadry ® | 4.50 |
| Isopropyl alcohol | q.s. |
| Purified water | q.s. |
| Enteric Coating on Extended-Release Cores | |
| Eudragit ® S 100 | 82.20 |
| Eudragit ® L 100 | 27.40 |
| Triethyl citrate | 32.90 |
| Talc | 11.00 |
| Acetone | q.s. |
| Isopropyl alcohol | q.s. |
| Purified water | q.s. |

### Timed Immediate-Release Cores:

| | quantity/capsule |
|---|---|
| Ingredients | (mg) |
| Eudragit ® S 100 | 29.40 |
| Eudragit ® L 100 | 9.80 |
| Triethyl citrate | 11.80 |
| Talc | 3.90 |
| Acetone | q.s. |
| Isopropyl alcohol | q.s. |
| Purified water | q.s. |

### Manufacturing Process:

1) Metoprolol succinate and Opadry® were dispersed in purified water and isopropyl alcohol.
2) The dispersion of step 1) was sprayed onto sugar spheres to form drug-coated cores.
3) Eudragit S 100, Eudragit L 100, triethyl citrate, and talc were dispersed in a mixture of acetone, isopropyl alcohol, and purified water to obtain an enteric coating dispersion.
4) Approximately 30% of the drug-coated cores of step 2) were coated with the dispersion of step 3) to obtain timed immediate-release cores.
5) Ethocel® 20 cps and Opadry® were dispersed in isopropyl alcohol and purified water.
6) The remaining part of the drug-coated cores of step 2) were coated with the dispersion of step 5) to obtain extended-release cores.
7) The extended-release cores of step 6) were coated with the enteric coating dispersion of step 3) to obtain timed extended-release cores.
8) The timed immediate-release cores of step 4) and the timed extended-release cores of step 7) were filled into suitable size capsule shells.

### Example 2

### Timed Extended-Release Cores

| | Quantity/Capsule |
|---|---|
| Ingredients | (mg) |
| Metoprolol succinate | 190.00 |
| Hydroxypropylmethyl cellulose | 40.00 |
| Sodium lauryl sulphate | 30.00 |
| Sugar spheres | 65.00 |
| Isopropyl alcohol | q.s. |
| Purified water | q.s. |
| Extended-Release Coating | |
| Ethocel ® 20 cps | 18.28 |
| Hydroxypropylmethyl cellulose | 2.43 |
| Triethyl citrate | 2.43 |
| Talc | 1.22 |
| Purified water | q.s. |
| Isopropyl alcohol | q.s. |
| Enteric Coating On Extended-Release Cores | |
| Eudragit ® S 100 | 39.24 |
| Triethyl citrate | 8.41 |
| Talc | 8.41 |
| Purified water | q.s. |
| Acetone | q.s. |
| Isopropyl alcohol | q.s. |

### Timed Immediate-Release Cores

| Ingredients | Quantity/Capsule (mg) |
|---|---|
| Eudragit ® S 100 | 39.24 |
| Triethyl citrate | 8.41 |
| Talc | 8.41 |
| Purified water | q.s. |
| Acetone | q.s. |
| Isopropyl alcohol | q.s. |

### Manufacturing Process:

1) Disperse metoprolol succinate, hydroxypropylmethyl cellulose, and sodium lauryl sulphate in a mixture of isopropyl alcohol and purified water.
2) Spray the dispersion of step 1) onto sugar spheres to form drug-coated cores.
3) Disperse Eudragit S 100, triethyl citrate, and talc into a mixture of acetone, isopropyl alcohol, and purified water to obtain an enteric coating dispersion.
4) Coat 50% of the drug-coated cores of step 2) with the enteric coating dispersion of step 3) to form timed immediate-release cores.
5) Disperse Ethocel® 20 cps, hydroxypropylmethyl cellulose, triethyl citrate, and talc in a mixture of isopropyl alcohol and purified water.
6) Coat the remaining part of the drug-coated cores of step 2) with the dispersion of step 5) to obtain extended-release cores.
7) Coat the extended-release pellets of step 6) with the enteric coating dispersion of step 3) to obtain timed extended-release cores.
8) Fill the timed immediate-release cores of step 4) and the timed extended-release cores of step 7) into suitable size capsule shells.

### Example 3

| Ingredients | Quantity/Capsule (mg) |
|---|---|
| Drug Coating-1 | |
| Metoprolol succinate | 133.00 |
| Hydroxypropylmethyl cellulose | 40.00 |
| Sodium lauryl sulphate | 30.00 |
| Sugar spheres | 65.00 |
| Isopropyl alcohol | q.s. |
| Purified water | q.s. |
| Extended-Release Coating | |
| Ethocel ® 20 cps | 22.78 |
| Hydroxypropylmethyl cellulose | 1.34 |
| Triethyl citrate | 1.34 |
| Talc | 1.34 |
| Purified water | q.s. |
| Isopropyl alcohol | q.s. |
| Drug Coating-2 | |
| Metoprolol succinate | 57.00 |
| Hydroxypropylmethyl cellulose | 17.10 |
| Sodium lauryl sulphate | 12.90 |
| Purified water | q.s. |
| Isopropyl alcohol | q.s. |
| Enteric Coating | |
| Eudragit ® S 100 | 57.27 |
| Eudragit ® L 100 | 57.27 |
| Triethyl citrate | 38.18 |
| Talc | 38.18 |
| Acetone | q.s. |
| Isopropyl alcohol | q.s. |
| Purified water | q.s. |

### Manufacturing Process:

1) Disperse metoprolol succinate, hydroxypropylmethyl cellulose, and sodium lauryl sulphate in a mixture of isopropyl alcohol and purified water.
2) Spray the dispersion of step 1 onto sugar spheres to obtain drug-coated cores.
3) Disperse Ethocel® 20 cps, hydroxypropylmethyl cellulose, triethyl citrate, and talc in isopropyl alcohol and purified water.
4) Coat the drug-coated cores of step 2) with the dispersion of step 3) to obtain extended-release cores.
5) Disperse metoprolol succinate, hydroxypropylmethyl cellulose, and sodium lauryl sulphate in a mixture of isopropyl alcohol and purified water.
6) Coat the extended-release cores of step 4) with the dispersion of step 5).
7) Disperse Eudragit® S 100, Eudragit® L 100, triethyl citrate, and talc in a mixture of acetone, isopropyl alcohol, and purified water to obtain an enteric coating dispersion.
8) Coat the coated cores of step 6) with the enteric coating dispersion of step 7) to obtain timed extended-release cores.
9) Fill the timed extended-release cores of step 8) into suitable size capsule shells.

### Example 4

### Timed Extended-Release Cores

| | Quantity/Capsule |
|---|---|
| Ingredients | (mg) |
| Seal Coating | |
| Sugar spheres | 65.00 |
| Ethocel ® 20 cps | 6.50 |
| Isopropyl alcohol | q.s. |
| Drug Coating | |
| Metoprolol succinate | 200.00 |
| Hydroxypropylmethyl cellulose | 40.00 |
| Purified water | q.s. |
| Extended-Release Coating | |
| Ethocel ® 20 cps | 35.04 |
| Hydroxypropylmethyl cellulose | 7.01 |
| Triethyl citrate | 2.34 |
| Talc | 2.34 |
| Isopropyl alcohol | q.s. |
| Purified water | q.s. |
| Enteric Coating on Extended-Release Cores | |
| Eudragit ® FS 30 D (on dried basis) | 112.84 |
| Talc | 11.28 |
| Simethicone | 1.25 |
| Purified water | q.s. |

### Timed Immediate-Release Cores

| Ingredients | Quantity/Capsule (mg) |
|---|---|
| Eudragit ® FS 30 D | 112.84 |
| Talc | 11.28 |
| Simethicone | 1.25 |
| Purified water | q.s. |

### Manufacturing Process:

1) Coat sugar spheres with a dispersion of Ethocel® in isopropyl alcohol.
2) Dissolve metoprolol succinate and hydroxypropylmethyl cellulose in purified water.
3) Spray the solution of step 2) onto the coated sugar spheres of step 1) to obtain drug-coated cores.
4) Disperse Eudragit® FS 30 D, talc, and simethicone in purified water to obtain an enteric coating dispersion.
5) Coat approximately 40% of the drug-coated cores of step 3) with the enteric coating dispersion of step 4) to obtain timed immediate-release cores.
6) Disperse Ethocel® 20 cps, hydroxypropylmethyl cellulose, triethyl citrate, and talc in isopropyl alcohol and purified water.
7) Coat the remaining part of the drug-coated cores of step 3) with the dispersion of step 6) to obtain extended-release cores.
8) Coat the extended-release pellets of step 7) with the enteric coating dispersion of step 4) to obtain timed extended-release cores.
9) Fill the timed extended-release cores of step 8) and timed immediate-release cores of step 5) into suitable size capsule shells.

### Example 5

### Timed Extended-Release Cores

| | Quantity/ |
|---|---|
| | Capsule |
| Ingredients | (mg) |
| Metoprolol succinate equivalent to 100 mg | 95.00 |
| metoprolol tartarate | |
| Opadry ® | 9.50 |
| Sugar spheres | 75.00 |
| Purified water | q.s. |
| Extended-Release Coating | |
| Ethocel ® 20 cps | 10.17 |
| Hydroxypropylmethyl cellulose | 1.79 |
| Triethyl citrate | 0.29 |
| Talc | 0.29 |
| Isopropyl alcohol | q.s. |
| Purified water | q.s. |
| Enteric Coating on Extended-Release Cores | |
| Eudragit ® S 100 | 40.72 |
| Eudragit ® L 100 | 13.57 |
| Triethyl citrate | 16.29 |
| Talc | 5.42 |
| Acetone | q.s. |
| Isopropyl alcohol | q.s. |
| Purified water | q.s. |
| Lubrication | |
| Talc | 2.14 |

### Timed Immediate-Release Cores

| Ingredients | Quantity/Capsule (Mg) |
|---|---|
| Eudragit ® S 100 | 15.86 |
| Eudragit ® L 100 | 5.29 |
| Triethyl citrate | 6.34 |
| Talc | 2.11 |
| Acetone | q.s. |
| Isopropyl alcohol | q.s. |
| Purified water | q.s. |
| Lubrication | |
| Talc | 0.84 |

### Manufacturing Process:

1) Metoprolol succinate and Opadry® were dispersed in purified water.
2) The dispersion of step 1) was sprayed onto sugar spheres to form drug-coated cores.
3) Eudragit® S 100, Eudragit® L 100, triethyl citrate, and talc were dispersed in a mixture of acetone, isopropyl alcohol, and purified water to obtain an enteric coating dispersion.
4) Approximately 30% of the drug-coated cores of step 2) were coated with the dispersion of step 3) to obtain timed immediate-release cores.
5) Ethocel® 20 cps, hydroxypropylmethyl cellulose, triethyl citrate, and talc were dispersed in isopropyl alcohol and purified water.
6) The remaining part of the drug-coated cores of step 2) was coated with the dispersion of step 5) to obtain extended-release cores.
7) The extended-release pellets of step 6) were coated with the enteric coating dispersion of step 3) to obtain timed extended-release cores.
8) The timed immediate-release cores of step 4) and the timed extended-release cores of step 7) were lubricated with talc and filled into suitable size capsule shells.

### Example 6

| | Quantity/ |
|---|---|
| | Capsule |
| Ingredients | (mg) |
| Metoprolol succinate equivalent to 100 mg | 95.00 |
| metoprolol tartarate | |
| Opadry ® | 9.50 |
| Sugar spheres | 75.00 |
| Purified water | q.s. |
| Extended-Release Coating | |
| Ethocel ® 20 cps | 11.29 |
| Hydroxypropylmethyl cellulose | 1.99 |
| Triethyl citrate | 0.33 |
| Talc | 0.33 |
| Isopropyl alcohol | q.s. |
| Purified water | q.s. |
| Enteric Coating on Extended-Release Cores | |
| Eudragit L 30 D-55 | 84.34 |
| Triethyl citrate | 8.43 |
| Talc | 12.65 |
| Purified water | q.s. |
| Lubrication | |
| Talc | 2.99 |

### Manufacturing Process:

1) Metoprolol succinate and Opadry® were dispersed in purified water.
2) The dispersion of step 1) was sprayed onto sugar spheres to form drug-coated cores.
3) Eudragit® L 30 D-55, triethyl citrate, and talc were dispersed in purified water to obtain an enteric coating dispersion.
4) Ethocel® 20 cps, hydroxypropylmethyl cellulose, triethyl citrate, and talc were dispersed in isopropyl alcohol and purified water.
5) The drug-coated cores of step 2) were coated with the dispersion of step 4) to obtain extended-release cores.
6) The extended-release pellets of step 5) were coated with the enteric coating dispersion of step 3) to obtain timed extended-release cores.
7) The timed extended-release cores of step 6) were lubricated with talc and filled into suitable size capsule shells.

### Example 7

The composition of Example 7 is the same as the composition of Example 6, but it comprises an additional seal coat layer on the sugar spheres.

| Ingredients | Quantity/Capsule (mg) |
|---|---|
| Seal Coated Inert Cores | |
| Sugar spheres | 68.18 |
| Ethocel ® 20 cps | 6.82 |
| Isopropyl alcohol | q.s. |
| Purified water | q.s. |

### Example 8

| | Quantity/ |
|---|---|
| | Capsule |
| Ingredients | (mg) |
| Metoprolol succinate equivalent to 100 mg | 95.00 |
| metoprolol tartarate | |
| Opadry ® | 09.50 |
| Sugar spheres | 75.00 |
| Purified water | q.s. |
| Extended-Release Coating | |
| Ethocel ® 20 cps | 14.55 |
| Hydroxypropylmethyl cellulose | 02.60 |
| Triethyl citrate | 0.43 |
| Talc | 0.43 |
| Isopropyl alcohol | q.s. |
| Purified water | q.s. |
| Enteric Coating on Extended-Release Cores | |
| Eudragit ® L 30 D-55 | 83.73 |
| Triethyl citrate | 08.37 |
| Talc | 12.56 |
| Purified water | q.s. |
| Lubrication | |
| Talc | 3.02 |

Example 8 was prepared following a similar process as given for Example 6.

### Dissolution Studies

Dissolution studies were carried out using capsules prepared according to Example 5, Example 6, Example 7, and Toprol-XL® tablet (100 mg). The dissolution studies were carried out in a USP type II apparatus, with a paddle rotation of 50 rpm, at a temperature of 37° C.±0.5° C., in 500 mL of 0.1N HCl for 4 hours, followed by changing the dissolution media to 900 mL of pH 7.5 phosphate buffer. The percentage of the drug released was calculated from the concentration of metoprolol succinate in the sample solutions collected at different time points and analyzed by an HPLC method using column Inertsil® ODS-3 and a mobile phase comprising sodium dihydrogen orthophosphate monohydrate. The results of the dissolution studies are provided in Table 1.

TABLE 1
Percentage release of Metoprolol in 500 mL of 0.1N HCl for 4 hours
followed by 900 mL of pH 7.5 phosphate buffer.

| | | Comparative | | | |
|---|---|---|---|---|---|
| | Cumulative | Example | Examples | | |
| Time (hours) | Time (hours) | Toprol-XL ® | 5 | 6 | 7 |
| Acid stage (0.1N HCl) | | | | | |
| 0 | 0 | 0 | 0 | 0 | 0 |
| 2 | 2 | — | 0 | — | — |
| 4 | 4 | 34 | 2 | 4 | 6 |
| Buffer stage (pH 7.5 | | | | | |
| Phosphate buffer) | | | | | |
| 1 | 5 | 44 | 32 | 16 | 20 |
| 2 | 6 | 49 | 41 | 27 | 32 |
| 4 | 8 | 59 | 55 | 45 | 49 |
| 8 | 12 | 75 | 75 | 68 | 72 |
| 12 | 16 | 88 | 84 | 81 | 87 |
| 16 | 20 | 95 | 90 | 89 | 96 |
| 20 | 24 | 99 | 93 | 93 | 100 |
| 24 | 28 | 100 | 100 | 97 | 103 |

The results of the dissolution studies with respect to the compositions of Example 5, Example 6, and Example 7 show that compositions of the present invention release not more than 15% of the total amount of drug in 0.1N HCl in 4 hours, and thereafter provide an extended-release of metoprolol for at least 20 hours in pH 7.5 phosphate buffer, wherein more than 70% of the drug is released in 20 hours to 24 hours. The currently available metoprolol extended-release Toprol-XL® tablet does not exhibit a lag time and around 34% of drug is released in 4 hours.

### Pharmacokinetic Parameters of Example 6 in Healthy Human Subjects

A pharmacokinetic study was conducted by orally administering to healthy human subjects, at about 9:00 p.m., metoprolol timed extended-release capsules (Example 6) and Toprol-XL® (100 mg) produced by AstraZeneca. The objective of this study was to show that the composition of Example 6 provides the desired characteristic of a timed extended-release pharmaceutical composition.

A single dose randomized, three treatment, three period, three sequence crossover study in healthy human subjects was carried out under fed condition to determine pharmacokinetic parameters.

TABLE 2
Pharmacokinetic parameter for Example 6 and Toprol-XL ® tablets

| | |
|---|---|
| Example 6 (Fed Evening) | Toprol-XL ® (Fed Evening) |

| Tₘₐₓ (hours) | T_{Lag} (hours) | Tₘₐₓ (hours) | T_{Lag} (hours) |
|---|---|---|---|
| 12.61 | 2.23 | 7.84 | 0.30 |
| | AUC₀₋ₜ | | AUC₀₋ₜ |
| Cₘₐₓ (ng/mL) | (ng · hr/mL) | Cₘₐₓ (ng/mL) | (ng · hr/mL) |
| 39.28 | 660.65 | 36.81 | 704.29 |

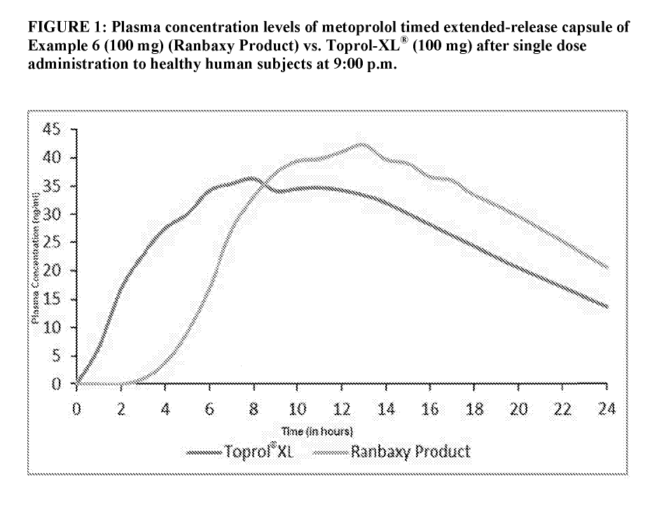

The results from these pharmacokinetic studies demonstrated distinctly different pharmacokinetic profiles for the two compositions under fed condition as shown in FIG. 1. The plasma concentration for Toprol-XL® achieved Tₘₐₓ at approximately 3 hours post-dosing while Example 6 had a lag time of 2.2 hours, and Tₘₐₓ at 12.6 hours post-dosing.

### Simulations for Determining Peak to Trough Ratio at Steady-State

Plasma concentration versus time curves obtained by administering a single dose of metoprolol timed extended-release capsule were simulated using WinNonlin® Version 5.3. The data obtained were projected to steady-state with a 24 hour dosing interval. Peak to trough ratio were estimated from the simulated Cₘₐₓ and Cₘₐₓ, obtained from the steady-state plasma. The data for steady state pharmacokinetic is given in Table 3.

TABLE 3
Simulated pharmacokinetic parameter values for Example 6

| Fed evening | Cₘₐₓ (ng/mL) | Cₘᵢₙ (ng/mL) | Peak to trough ratio |
|---|---|---|---|
| Example 6 | 58.39 | 21.42 | 2.73 |

The data obtained from the in-vitro as well as in-vivo studies (including simulation study) indicates that the pharmacokinetic parameters of the present invention provide the desired characteristics of the timed extended-release pharmaceutical composition.

We claim:

1. A timed extended-release pharmaceutical composition comprising:
a) a core comprising metoprolol;
b) an extended-release coating comprising an extended-release polymer; and
c) at least one outer coating layer comprising an enteric polymer
wherein the pharmaceutical composition exhibits the following in-vitro dissolution profile, when measured in United States Pharmacopeia (USP) type 2 dissolution apparatus, with a paddle rotation of 50 rpm, at a temperature of 37° C.±0.5° C. in 500 mL of 0.1N HCl for 4 hours, followed by 900 mL of pH 7.5 phosphate buffer: (a) not more than about 15% of the total amount of metoprolol is released in 4 hours; and (b) more than 70% of the total amount of metoprolol is released in 20 hours to 24 hours.

2. The timed extended-release pharmaceutical composition according to claim 1, further comprising a timed immediate-release pharmaceutical composition.

3. The timed extended-release pharmaceutical composition according to claim 2, wherein the timed immediate-release pharmaceutical composition comprises:
a) a core comprising metoprolol; and
b) an outer coating comprising an enteric polymer.

4. The timed extended-release pharmaceutical composition according to claim 1, wherein the core is a matrix core comprising metoprolol and optionally one or more pharmaceutically acceptable excipients.

5. The timed extended-release pharmaceutical composition according to claim 1, wherein the core is a coated core comprising:
a) an inert bead;
b) optionally, a seal coat layer over the inert bead; and
c) a coating layer comprising metoprolol over the inert bead of step a) or the coated bead of step b).

6. The timed extended-release pharmaceutical composition according to claim 1, wherein the extended-release coating is about 5% to about 15% based on the weight of the drug coated core.

7. The timed extended-release pharmaceutical composition according to claim 1, wherein the extended-release polymer is a water-soluble polymer, a water-insoluble polymer, or a mixture thereof.

8. The timed extended-release pharmaceutical composition according to claim 7, wherein the water-soluble polymer is selected from the group comprising hydroxypropylmethyl cellulose, hydroxyethyl cellulose, polyethylene glycol, poly(ethylene oxide), hydroxypropyl cellulose, carboxymethyl cellulose, xanthan gum, starch, polyvinyl pyrrolidone, or mixtures thereof.

9. The timed extended-release pharmaceutical composition according to claim 7, wherein the water-insoluble polymer is selected from the group comprising cellulose ethers, cellulose esters, polymethacrylic acid esters copolymers, aminoalkyl methacrylate copolymers, copolymers of polyvinyl acetate and polyvinyl pyrrolidone, or mixtures thereof.

10. The timed extended-release pharmaceutical composition according to claim 9, wherein the cellulose ether is ethyl cellulose.

11. The timed extended-release pharmaceutical composition according to claim 1, wherein the extended-release coating comprises a water-insoluble polymer and a pore-former.

12. The timed extended-release pharmaceutical composition according to claim 11, wherein the pore-former is selected from the group comprising low viscosity grade hydroxypropylmethyl cellulose, sodium alginate, sugars and sugar alcohols, low molecular weight polyethylene glycol, polyvinyl alcohol, polyvinyl pyrrolidone, or mixtures thereof.

13. The timed extended-release pharmaceutical composition according to claim 11, wherein the water-insoluble polymer and the pore-former are present in a ratio of about 50:50 to about 99:1.

14. The timed extended-release pharmaceutical composition according to claim 13, wherein the water-insoluble polymer and the pore-former are present in a ratio of about 75:25 to about 95:5.

15. The timed extended-release pharmaceutical composition according to claim 1, wherein the enteric polymer is selected from the group comprising hydroxypropylmethyl cellulose acetate succinate, hydroxypropylmethyl cellulose phthalate, cellulose acetate phthalate, methacrylic acid copolymer, or mixtures thereof.

16. The timed extended-release pharmaceutical composition according to claim 15, wherein the enteric polymer is one or more methacrylic acid copolymer selected from the group comprising Eudragit® L 30 D-55, Eudragit® L 100-55, Eudragit® L 100, Eudragit® S 100, Eudragit® L 12,5, Eudragit® S 12,5, or Eudragit® FS 30 D.

17. The timed extended-release pharmaceutical composition according to claim 16, wherein the methacrylic polymer is Eudragit® L 30 D-55.

18. The timed extended-release pharmaceutical composition according to claim 16, wherein the methacrylic polymer is Eudragit® FS 30 D.

19. The timed extended-release pharmaceutical composition according to claim 16, wherein the methacrylic polymer is Eudragit® S 100.

20. The timed extended-release pharmaceutical composition according to claim 16, wherein the methacrylic polymer is Eudragit® L 100.

21. The timed extended-release pharmaceutical composition according to claim 16, wherein the methacrylic polymer is a combination of Eudragit® S 100 and Eudragit® L 100.

22. The timed extended-release pharmaceutical composition according to claim 21, wherein Eudragit® S 100 and Eudragit® L 100 are present in a ratio of about 1:1 to about 5:1.

23. A timed extended-release pharmaceutical composition comprising:
a) a core comprising metoprolol;
b) an extended-release coating comprising an extended-release polymer; and
c) at least one outer coating comprising an enteric polymer
wherein the pharmaceutical composition provides a Tₘₐₓ of more than 8 hours when administered to healthy human subjects.

24. The timed extended-release pharmaceutical composition according to claim 23 wherein the pharmaceutical composition further provides an in-vivo lag time of at least 2 hours.
